(19) United States
(12) Patent Application Publication
(10) Pub. No.: US 2023/0100890 A1
(43) Pub. Date: Mar. 30, 2023

(54) USE OF COMBINED INHALANT CANNABINOID THERAPY IN THE TREATMENT OF MIGRAINE

(71) Applicant: Medicinal Cannabis of Georgia, Augusta, GA (US)

(72) Inventor: Babak Baban, Augusta, GA (US)

(21) Appl. No.: 17/930,218

(22) Filed: Sep. 7, 2022

Related U.S. Application Data
(60) Provisional application No. 63/241,207, filed on Sep. 7, 2021.

(51) Int. Cl. A61K 9/00; A61K 31/05; A61K 31/352

(52) U.S. Cl. CPC A61K 9/0073; A61K 31/05; A61K 31/352; A61K 9/0075

(57) ABSTRACT

In one aspect, the disclosure relates to pharmaceutical compositions comprising one or more cannabinoids and methods of treating and preventing migraine using the same. In one aspect, the pharmaceutical compositions can be formulated for administration by inhalation in order to rapidly access endogenous cannabinoid receptors while avoiding systemic side effects associated with oral or other routes of administration.

### CROSS-REFERENCE TO RELATED APPLICATIONS

This application claims the benefit of and priority to co-pending U.S. Provisional Patent Application No. 63/241,207, filed on Sep. 7, 2021, the contents of which are incorporated by reference herein in their entireties.

### BACKGROUND

Migraine or migraine headache is a recurring headache that can be induced by a number of triggers, which may be different from person to person. Common triggers can include tobacco use, caffeine, changes to or overuse of medication, stress and anxiety, flashing or bright lights, sleep disruptions, hormonal changes, weather changes, physical activity, loud noises, strong smells, skipping meals, or consumption of specific foods, especially in combination (e.g., alcohol, aged cheeses, fermented foods, monosodium glutamate, chocolate, and cured or processed meats). Migraines may have a genetic cause or component and typically have different phases starting up to 24 hours prior to headache onset. A migraine may be felt on one side of the head and can be associated with throbbing pain, increased sensitivity to external stimuli such as light and noise, nausea and/or vomiting, and the like.

Management of migraine may include prevention strategies such as hormone therapy for women, logging symptoms and triggers in order to avoid the same, practicing stress management techniques, or taking certain nutritional supplements (e.g., vitamin B2 or coenzyme Q10). Treatment of migraine includes resting in dark, quiet rooms, drinking fluids, and taking certain pain relievers and/or other drugs. However, no cure for migraine exists.

Cannabinoids are known to have anticonvulsive, analgesic, antiemetic, and anti-inflammatory properties and are thus promising as acute and prophylactic treatment of migraine pain. The body has numerous endogenous cannabinoid receptors including CB1 receptors in the brain and CB2 receptors elsewhere in the body. CB1 receptors represent an attractive target for treatment of migraine via blocking peripheral and central nociceptive traffic and reducing pathologically enhanced central excitability predisposing migraine sufferers to cortical spreading depolarization (CSD). Meanwhile, CB2 receptors in immune cells may present attractive druggable targets for reducing inflammation associated with severe forms of migraine. In one aspect, administration of exogenous compounds lacking unwanted peripheral pro-nociceptive components or endogenous cannabinoids generated via inhibited degradation pathways and combined with other supportive agents presents an attractive strategy for treatment and prevention of migraines.

Despite advances in migraine treatment research, there is still a scarcity of compounds and compositions that are both potent and efficacious in the treatment of acute migraine symptoms while also being effective in the prevention of migraine. Ideally the compositions would be deliverable via a method that can be administered by patients at home, but that avoids gastrointestinal degradation of compounds and/or systemic side effects that may be associated with oral dosage forms. These needs and other needs are satisfied by the present disclosure.

### SUMMARY

In accordance with the purpose(s) of the present disclosure, as embodied and broadly described herein, the disclosure, in one aspect, relates to pharmaceutical compositions comprising one or more cannabinoids and methods of treating and preventing migraine using the same. In one aspect, the pharmaceutical compositions can be formulated for administration by inhalation in order to rapidly access endogenous cannabinoid receptors while avoiding systemic side effects associated with oral or other routes of administration.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### DETAILED DESCRIPTION

In one aspect, disclosed herein are pharmaceutical compositions including (I) at least one cannabinoid or a pharmaceutically acceptable salt or ester thereof and (II) a pharmaceutically-acceptable carrier, wherein the composition is capable of being administered to a subject by inhalation.

Also disclosed herein is a method for treating or preventing at least one symptom associated with migraine in a subject, the method including the step of administering to the subject a disclosed pharmaceutical composition. In another aspect, the at least one symptom includes sensitivity to light, sensitivity to sound, nausea, vomiting, pain, weakness, numbness, vision loss, difficulty speaking, visual hallucinations, or any combination thereof. In any of these aspects, the subject can be a human.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a cannabinoid,” “a pharmaceutical composition,” or “an excipient,” include, but are not limited to, mixtures or combinations of two or more such cannabinoids, pharmaceutical compositions, or excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a cannabinoid refers to an amount that is sufficient to achieve the desired improvement in the property modulated by the formulation component, e.g. achieving the desired level of migraine relief. The specific level in terms of wt% in a composition required as an effective amount will depend upon a variety of factors including the amount and type of cannabinoids, amount and type of other active ingredients, amount and type of thermally excipients, and dosage frequency for the pharmaceutical composition that includes the cannabinoids.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as migraines or other headaches. The effect can be therapeutic in terms of a partial or complete cure of a disease, condition, symptom or adverse effect attributed to the disease, disorder, or condition. The term “treatment” as used herein can include any treatment of migraines in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

An “isotonic” nasal spray or nebulizer solution has the same salt concentration as the human body. Meanwhile, a “hypertonic” nasal spray or nebulizer solution has a slightly higher concentration of salt than in the human body. Isotonic and/or hypertonic solutions are comfortable for nasal and inhaled applications of pharmaceutically active ingredients. In one aspect, isotonic and hypertonic solutions, especially when administered as nasal washes, may also have the capability to clear mucus from the nasal and/or sinus passages.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

In various aspects, it is contemplated herein that the disclosed compounds further comprise their biosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and biolsosterism in drug design”; Prog. Drug Res. 1991, 37, 287-371; (iii) Burger A, “Isosterism and bioanalogy in drug design”, Med. Chem. Res, 1994, 4, 89-92; (iv) Clark R D, Ferguson A M, Cramer R D, “Bioisosterism and molecular diversity”, Perspect. Drug Discovery Des. 1998, 9/10/11, 213-224; (v) Koyanagi T, Haga T, “Bioisosterism in agrochemicals”, ACS Symp. Ser. 1995, 584, 15-24; (vi) Kubinyi H, “Molecular similarities. Part 1. Chemical structure and biological activity”, Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem, Rev. (Washington, D.C.) 1996, 96, 3147-3176; (ix) Soskic V, Joksimovic J, “Bioisosteric approach in the design of new dopaminergic/serotonergic ligands”, Curr. Med. Chem. 1998, 5, 493-512 (x) Thornber C W, “Isosterism and molecular modification in drug design”, Chem. Soc. Rev. 1979, 8, 563-80.

In further aspects, bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters, amides or carboxylic acids are compounds containing two sites for hydrogen bond acceptance. In one embodiment, the ester, amide or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulfuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quatemized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

As used herein, “cortical spreading depolarization” or “CSD” refers to a series of events following a neurological injury. These events may include a loss of ion homeostasis, an altered vascular response, changes in synapses, and/or alterations in electrical activity in the brain. In some aspects, migraine patients can experience CSD.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration via inhalation.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids that can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection, inhalation, or parenteral administration. Carriers include solids such as, for example, dry powders, or liquids, such as, for example, liquid carriers that can be aerosolized with a suitable propellant and inhaler mechanism, and the type of carrier is chosen based on the type of administration being used. The compounds may be administered as a dosage that has a known quantity of the compound.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In one aspect, pharmaceutical compositions for administration via the pulmonary route (e.g., by inhalation), including by nebulization, according to the present disclosure can include compounds useful for adjusting a solution's osmotic pressure (tonicity). In a further aspect, these compounds can include, but are not limited to, sodium chloride, dextrose, and combinations thereof. In some aspects, these compounds adjust osmotic pressure to a range of from about 300 to about 700 mOsmol/kg, or about 300, 350, 400, 450, 500, 550, 600, 650, or about 700 mOsmol/kg, or a combination of any of the foregoing values, or a range encompassing any of the foregoing values.

In another aspect, the pharmaceutical compositions can include compounds useful for adjusting solution pH. Without wishing to be bound by theory, a liquid medication, whether inhaled or injected, may work best when the carrier has a pH similar to physiological conditions. In another aspect, pH adjustments may also enhance drug stability and/or drug solubility. In one aspect, pH can be adjusted by adding a compound such as sodium hydroxide or potassium hydroxide (to increase the pH and/or made the carrier more basic) or hydrochloric acid or sulfuric acid (to decrease the pH and/or make the carrier more acidic). In another aspect, pH can be adjusted using a biocompatible buffer such as, for example, 2-(N-morpholino)ethanesulfonic acid (MES), bis-tris methane, N-(2-acetamido)-2-iminodiacetic acid (ADA), N-(2-acetamido)-2-aminoethanesulfonic acid (ACES), bis-tris propane, piperazine-N,N′-bis(2-ethanesulfonic acid (PIPES), N-(2-acetamido)-2-am inoethanesulfonic acid (ACES), 2-hydroxy-3-morpholinopropanesulfonic acid (MOPSO), cholamine chloride, 3-(N-morpholino)propanesulfonic acid (MOPS), N,N-bis(2-hydroxyethyl)-2-aminoethanesulfonic acid (BES), 2-[(2-hydroxy-1,1-bis(hydroxymethyl)ethyl)amino]ethanesulfonic acid (TES), 4-(2-hydroxyethyl)-1-piperazineethanesulfonic acid (HEPES), 3-(N, N-bis[2-hydroxyethyl]amino)-2-hydroxypropanesulfonic acid (DIPSO), 4-(N-morpholino)butanesulfonic acid (MOBS), acetamidoglycine, 2-hydroxy-3-[tris(hydroxymethyl)methylamino]-1]propanesulfonic acid (TAPSO), triethylammonium acetate (TEAA), piperazine-1,4-bis(2-hydroxypropanesulfonic acid) dihydrate (POPSO), N-(hydroxyethyl)piperazine-N′-2-hydroxypropanesulfonic acid (HEPPSO), 4-(2-hydroxyethyl)-1-piperazinepropanesulfonic acid (EPPS), tricine, tris, glycinamide, glycylglycine, N-(2-hydroxyethyl)piperazine-N′-(4-butanesulfonic acid) (HEPBS), bicine, [tris(hydroxymethyl)methylamino]propanesulfonic acid (TAPS), N-cyclohexyl-2-aminoethanesulfonic acid (CHES), aminomethyl propanol (AMP), N-(1,1-dimethyl-2-hydroxyethyl)-3-amino-2-hydroxypropanesulfonic acid (AMPSO), N-cyclohexyl-2-hydroxyl-3-aminopropanesulfonic acid (CAPSO), N-cyclohexyl-3-aminopropanesulfonic acid (CAPS), 4-(cyclohexylamino)-1-butanesulfonic acid (CABS), citrate buffer, phosphate buffer, a sodium salt thereof, or a combination thereof. In some aspects, a pH of from about 4.5 to about 6.5 may be useful to prevent sneezing or respiratory irritation. In another aspect, pH may also be important to product stability during transport and/or storage.

In one aspect, the formulations can be purged with an inert gas such as, for example, nitrogen, during the packaging process. In one aspect, purging may help prevent or reduce oxidation, thus extending the shelf life of the pharmaceutical compositions. In another aspect, the formulations can include a preservative such as, for example, benzalkonium chloride or another quaternary ammonium compound, ethanol, propylene glycol, benzoyl alcohol, chlorobutanol, methylparaben or another paraben, or a combination thereof. In any of these aspects, the preservative can reduce or prevent microbial growth in the pharmaceutical compositions. In still another aspect, the pharmaceutical compositions can include a surfactant such as, for example, polysorbate 20, polysorbate 80, or another surfactant. In another aspect, the surfactant can increase suspendability of the active ingredient and/or stability of suspensions including the active ingredient. In some aspects, the formulations can include a chelating agent such as, for example, disodium EDTA, which can, in a further aspect, increase the stability of the formulations. In one aspect, the formulations can include a suspending agent. In a further aspect, the suspending agent can be carboxymethyl cellulose or sodium carboxymethyl cellulose. In one aspect, the suspending agent can alter the viscosity of the solution. In another aspect, if the active ingredient is administered as a suspension, the suspending agent can enhance the suspendability of the active ingredient and/or the stability of any suspensions formed using the active ingredient. In some aspects, the formulations include a co-solvent such as, for example, alcohol, a polyethylene glycol such as, for example, PEG 400, propylene glycol, or a combination thereof. In another aspect, the co-solvent can increase the solubility of the active ingredient(s). In one aspect, the formulations can include a humectant such as, for example, glycerin. In one aspect, for pharmaceutical compositions intended to be administered by nebulizer, doses can be packaged as unit-dose vials to avoid the need for antimicrobial agents. Further in this aspect, the compositions are sterile.

In another aspect, pharmaceutical compositions administered by the pulmonary route, e.g., by a metered dose inhaler (MDI), can include antioxidants such as, for example, acetone sodium bisulfate, ascorbic acid, or a combination thereof. In another aspect, the pharmaceutical compositions for use in MDIs disclosed herein can include preservatives such as, for example, ammonia, benzalkonium chloride, cetylpyridinium chloride, and other quaternary ammonium compounds, chlorobutanol, methylparaben, propylparaben, and other parabens, sodium metabisulfite, sodium bisulfite, sodium sulfite, thymol, or a combination thereof. In one aspect, the compositions disclosed herein may include agents for adjusting tonicity or osmotic pressure including, but not limited to, sodium chloride, sodium sulfate, and combinations thereof. In one aspect, the formulations may include flavoring agents including, but not limited to, citric acid, menthol, saccharin, saccharin sodium dehydrate, or a combination thereof. In any of these aspects, the formulations can include a chelating agent such as, for example, disodium EDTA. In one aspect, formulations for administration by metered dose inhaler include a cosolvent such as, for example, ethanol, glycerin, propylene glycol, water, or a combination thereof. In one aspect, the formulations disclosed herein include buffering agents and/or pH stabilizers including, but not limited to, glycine, hydrochloric acid, lysine monohydrate, nitric acid, sodium bisulfate, sodium citrate, sodium hydroxide, sulfuric acid, tromethamine, another biocompatible pH stabilizer or buffering agent, or a combination thereof. In one aspect, the excipients in the MDI formulations can include a dispersion or solubilization agent such as, for example, lecithin, magnesium stearate, oleic acid, a polyethylene glycol (e.g., PEG 10000), sorbitan trioleate, carboxylic acid functionalized methyl polyethylene glycol (f-mPEG), oligolectic acid (OLA), a combination thereof, or any of these co-formulated with ethanol. In one aspect, a suspending aid such as, for example, polysorbate 80 or polyvinylpyrrolidone K25 can be included in the formulations disclosed herein. In another aspect, in some formulations disclosed herein, a surfactant can be useful. Further in this aspect, the surfactant can be selected from sorbitan monolaurate, sorbitan monooleate, sorbitan trioleate, a PEG (e.g., PEG300, PEG 600, PEG 1000), a propoxylated PEG, PEG-4 lauryl ether, PEG-23 lauryl ether, PEG-10 cetyl ether, PEG-2 stearyl ether, PEG-10 oleyl ether, polysorbate 20, polysorbate 60, polysorbate 80, a polypropylene glycol (PPG) such as PPG 2000, a block copolymer of PEG and PPG such as, for example, pluronic 10-R5, 17-R2, 17-R4, 25-R4, F-68, F-127, L043, L-44 NF, L-62, L-64, L-101, sodium dioxtyl sulfosuccinate, or a combination thereof. In some aspects, these excipients can have multiple functions, e.g., benzalkonium chloride may aid in wetting and solubilization in addition to acting as a preservative. In one aspect, when the MDI formulation is a suspension, a bulking agent may be included. In one aspect, the bulking agent can be a saccharide such as lactose or maltose, an amino acid such as glycine or leucine, a salt, or a combination thereof.

In one aspect, when preparing the cannabinoids as disclosed herein for administration by an MDI, these dry components should be reduced to have an average particle size suitable for administration by MDI. In one aspect, particle size reduction can be achieved by milling (e.g., ball milling or jet milling), spray drying, use of a supercritical fluid, or a combination thereof.

### Pharmaceutical Compositions and Methods of Dispensing

In one aspect, disclosed herein is a pharmaceutical composition including one or more cannabinoids and a pharmaceutically-acceptable carrier. In one aspect, the cannabinoids can be administered in the same treatment and/or co-packaged in the same unit dose vial such as, for example, in a liquid formulation or suspension that can be aerosolized.

In one aspect, the cannabinoid can include Δ⁹-tetrahydrocannabinol (THC) and cannabidiol (CBD). Further in this aspect, the THC can have a unit dose of from about 1 to about 6 mg, or of about 1, 1.5, 2, 2.5, 3, 3.5, 4, 4.5, 5, 5.5, or about 6 mg, or a combination of any of the foregoing values, or a range encompassing any of the foregoing values. In another aspect, the cannabidiol can have a unit dose of from about 1 mg to about 5 mg, or of about 1, 1.5, 2, 2.5, 3, 3.5, 4, 4.5, or about 5 mg, or a combination of any of the foregoing values, or a range encompassing any of the foregoing values. In one aspect, the composition can include 3 mg of THC and 2.5 mg of CBD.

In one aspect, in the pharmaceutical composition the Δ⁹-tetrahydrocannabinol and the cannabidiol are present in a weight ratio of from about 1:5 to about 5:1, or of about 1:5, 1:4, 1:3, 1:2, 2:1, 3:1, 4:1, 2:5, 3:5, 4:5, 1:1, 5:4, 5:3, 5:2, 5:1, or about 3:2.5, or a combination of any of the foregoing values, or a range encompassing any of the foregoing values.

In one aspect, the cannabinoid can include Δ⁹-tetrahydrocannabinol (THC) and cannabichromene (CBC). Further in this aspect, the THC can have a unit dose of from about 1 to about 6 mg, or of about 1, 1.5, 2, 2.5, 3, 3.5, 4, 4.5, 5, 5.5, or about 6 mg, or a combination of any of the foregoing values, or a range encompassing any of the foregoing values. In another aspect, the cannabichromene can have a unit dose of from about 1 mg to about 5 mg, or of about 1, 1.5, 2, 2.5, 3, 3.5, 4, 4.5, or about 5 mg, or a combination of any of the foregoing values, or a range encompassing any of the foregoing values. In one aspect, the composition can include 3 mg of THC and 2.3 mg of CBC.

In one aspect, in the pharmaceutical composition, the Δ⁹-tetrahydrocannabinol and the cannabichromene are present in a weight ratio of from about 1:5 to about 5:1, or of about 1:5, 1:4, 1:3, 1:2, 2:1, 3:1, 4:1, 2:5, 3:5, 4:5, 1:1, 5:4, 5:3, 5:2, 5:1, or about 3:2.3, or a combination of any of the foregoing values, or a range encompassing any of the foregoing values.

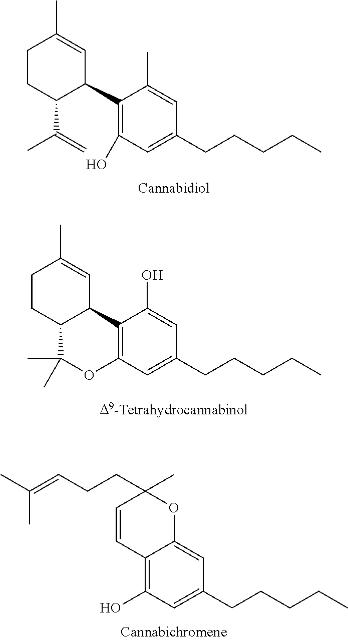

In one aspect, the pharmaceutically acceptable salt can be an organic salt, a metal salt, or any combination thereof. In another aspect, the pharmaceutically acceptable salt can be NH⁴⁺, Na⁺, Li⁺, K⁺, Ca²⁺, Mg²⁺, Fe²⁺, Fe²⁺, Cu²⁺, Al³⁺, Zn²⁺, 2-trimethylethanolammonium cation (choline), or a quaternary salt of isopropylamine, trimethylamine, diethylamine, triethylamine, tripropylamine, ethanolamine, 2-dimethylaminoethanol, 2-diethylaminoethanol, lysine, arginine, histidine, or any combination thereof. In another aspect, the pharmaceutically acceptable ester can be a prodrug.

In one aspect, the pharmaceutical composition includes peppermint oil, peppermint extract, or any combination thereof. In one aspect, the pharmaceutical composition does not include caffeine. In another aspect, the composition includes particles having an average diameter of 9 μm or less, or of 9, 8.5, 8, 7.5, 7, 6.5, 6, 5.5, or of 5 μm or less, or is provided as a solution with only a liquid phase and no particles.

In another aspect, in the pharmaceutical composition can be a spray comprising from about 20 mg to about 60 mg of ethanol per 100 μL of spray, or about 20, 25, 30, 35, 40, 45, 50, 55, or about 60 mg of ethanol per 100 μL of spray, or a combination of any of the foregoing values, or a range encompassing any of the foregoing values.

In still another aspect, the pharmaceutical composition can be a spray including:
a. Δ⁹-tetrahydrocannabinol in an amount of from about 1 mg to about 5 mg per 100 μL of spray; and
b. cannabidiol or cannabichromene in an amount of from about 1 mg to about 5 mg per 100 μL of spray.

In yet another aspect, the pharmaceutical composition can be a spray including:
a. Δ⁹-tetrahydrocannabinol in an amount of from about 1 mg to about 5 mg per 100 μL of spray;

b. cannabidiol or cannabichromene in an amount of from about 1 mg to about 5 mg per 100 μL of spray; and
c. ethanol in an amount of from about 20 mg to about 60 mg per 100 μL of spray.

In one aspect, the pharmaceutical composition can include a peppermint oil or extract. In another aspect, the peppermint oil or extract can be obtained from Mentha×piperita by any method known in the art including, but not limited to, azeotropic distillation (e.g., hydrodistillation, hydrodiffusion, and steam distillation) and/or solvent extraction. In one aspect, the peppermint oil or extract contains a significant proportion of menthol. Without wishing to be bound by theory, in one aspect, peppermint oil or extract and/or menthol may enhance blood flow and/or oxygen flow in or around the sinuses, thereby both enhancing the effect of the cannabinoids in the pharmaceutical compositions and providing some additional symptom relief. In still another aspect, peppermint oil or extract may cause a cooling sensation which can bring additional migraine pain relief.

In some aspects, the pharmaceutically acceptable carrier can be an inhalable dry powder. In one aspect, when the pharmaceutically acceptable carrier is a dry powder, it can include glucose, arabinose, maltose, saccharose, dextrose, lactose, mannitol, maltitol, lactitol, sorbitol, or a combination thereof. In another aspect, the carrier particles can have an average particle diameter of from about 0.5 to about 500 μm, or of about 0.5, 1, 2, 3, 4, 5, 6, 7, 8, 9, 10, 20, 30, 40, 50, 100, 150, 200, 250, 300, 350, 400, 450, or about 500 μm, or a combination of any of the foregoing values, or a range encompassing any of the foregoing values. In another aspect, the pharmaceutical compositions disclosed herein delivered by dry powder inhaler do not contain any carriers or excipients. Further in this aspect, aggregate dry powder active ingredients can be converted into an aerosol by airflow during inspiration.

In other aspects, the pharmaceutically acceptable carrier can be saline. In some aspects, if the solution or composition to be nebulized is isotonic or mildly hypotonic, the pharmaceutically acceptable carrier can be sterile water.

In one aspect, the pharmaceutical composition can be formulated as a spray having a volume of about 100 μL. Further in this aspect, the pharmaceutically acceptable carrier can include or can be ethanol. In one aspect, when ethanol is present, it is present in a volume of about 51 μL ethanol per 100 μL of spray.

In one aspect, the pharmaceutically acceptable carrier can be a high vapor pressure propellant. In another aspect, the pharmaceutically acceptable carrier can be a hydrofluoroalkane (HFA) such as, for example, HFA 134a (1,1,1,2-tetrafluoroethane), HFA 227 (1,1,1,2,3,3,3-heptafluoropropane), HFA 152a (1,1-difluoroethane), or a combination thereof. In an alternative aspect, the pharmaceutically acceptable carrier can be isobutane. In still another aspect, the pharmaceutically acceptable carrier can be a hydrofluoroolefin (HFO) such as, for example, HFO 1234ze (trans-1,3,3,3-tetrafluoroprop-1-ene), HFO 1234yf (2,3,3,3-tetrafluoroprop-1-ene), or a combination thereof.

In one aspect, disclosed herein is a metered dose inhaler or a dry powder inhaler that includes the pharmaceutical compositions disclosed herein. In an alternative aspect, disclosed herein is a nebulizer ampoule that includes the pharmaceutical compositions disclosed herein. In one aspect, nebulizers are advantageous in that large doses of drug can be administered while the patient takes multiple breaths and can be used by patients of any age as breathing does not need to be coordinated with dispensation (as with a metered dose inhaler). In another aspect, nebulizers do not require propellants. However, in some aspects, gas flow such as air or oxygen may be required for aerosolization. In one aspect, the flow can be from about 3.5 to about 8 L/min, or can be about 3.5, 4, 4.5, 5, 5.5, 6, 6.5, 7, 7.5, or about 8 L/min, or a combination of any of the foregoing values, or a range encompassing any of the foregoing values. In another aspect, flow rate can be selected to achieve the desired droplet characteristics (e.g., average particle diameter).

In one aspect, the nebulizer can be a jet nebulizer, an ultrasonic nebulizer, or a mesh nebulizer. In a further aspect, when the nebulizer is a jet nebulizer, it is associated with lower equipment cost and may be capable of delivering drugs that are less effective using a metered dose inhaler. In another aspect, jet nebulizers leave less medication behind as waste than other dosage forms. In some aspects, a jet nebulizer requires pressurized gas to withdraw medication from a reservoir. In some aspects, the jet nebulizer can be a breath-enhanced jet nebulizer. In one aspect, with a breath-enhanced jet nebulizer, more aerosols can be released during inhalation due to negative pressure created by inspiration. In an alternative aspect, the jet nebulizer can be a breath-actuated jet nebulizer. In one aspect, a breath-actuated jet nebulizer is configured to sense breath intake and deliver aerosol only at inspiration.

In one aspect, when the nebulizer is an ultrasonic nebulizer, it may be more efficient at drug delivery than a jet nebulizer. However, in another aspect, an ultrasonic nebulizer may not be able to effectively aerosolize viscous solutions. In some aspects, an ultrasonic nebulizer should not be used with a suspension or a protein.

In still another aspect, when the nebulizer is a mesh nebulizer, it may produce less noise than a jet or ultrasonic nebulizer. In another aspect, a mesh nebulizer has a plate with a plurality of holes, or a mesh, which can be vibrated to produce an aerosol. Further in this aspect, the pharmaceutical composition can be placed above the mesh or plate. In another aspect, when the mesh or plate vibrates, a pumping action begins that extrudes the pharmaceutical composition through the mesh or plate as aerosols, wherein the aerosol particle size is determined by the diameter of the holes or mesh screen.

In any of these aspects, an aerosol particle size of less than about 9 μm, or of less than about 5 μm is created by the nebulizer or inhaler. In some aspects, the particle size can be about 2, 2.5, 3, 3.5, 4, 4.5, 5, 5.5, 6, 6.5, 7, 7.5, 8, 8.5, or about 9 μm, or a combination of any of the foregoing values, or a range encompassing any of the foregoing values. In any of these aspects, average treatment time with a nebulizer can be from about 5 min to about 8 min, or can be about 5, 6, 7, or about 8 min, or a combination of any of the foregoing values, or a range encompassing any of the foregoing values. In one aspect, average active ingredient output for a nebulizer can be from about 400 to about 500 mg/min, or can be about 400, 410, 420, 430, 440, 450, 460, 470, 480, 490, or about 500 mg/min, or a combination of any of the foregoing values, or a range encompassing any of the foregoing values. In another aspect, when two or more active ingredients are included in the compositions, the active ingredients can be delivered by the nebulizer at different rates.

In another aspect, the pharmaceutical compositions can be delivered nasally. In one aspect, nasal administration can be accomplished using a nebulizer as disclosed previously accompanied by a mask that can be fitted to the face of a subject to enable nasal breathing. In another aspect, nasal delivery of medications can be more effective at reaching the sinuses as well as, in some cases, the upper airways, than inhaled or nebulized medication. In an alternative aspect, nasal sprays and other nasal delivery mechanisms are inexpensive, portable, and easy to use and may help ensure patient compliance with treatment. In one aspect, nasal delivery can be accomplished using a pressurized metered-dose inhaler or a metered-dose spray pump. In some aspects, delivery location of nasally administered compositions is affected by particle size. In one aspect, particles larger than about 10 μm remain primarily in the nose and sinuses, whereas particles smaller than about 9 μm are capable of traveling to the upper airways and into the lungs.

In one aspect, when the pharmaceutical compositions are delivered nasally, they can be aqueous solutions, suspensions, powders, gels, and/or emulsions. In another aspect, when the pharmaceutical compositions are delivered via metered-dose spray pumps or via other multi-dose containers (e.g., side-actuated spray pumps), the pharmaceutical compositions can include a preservative such as, for example, benzalkonium chloride. In some aspects, nasally delivered pharmaceutical compositions may be packaged as drops, as compositions to be vaporized (e.g., menthol vapor inhalers for the common cold), as breath powered or hand-actuated spray pumps or drop dispensers, in electrically powered nebulizers or atomizers, by hand-actuated powder sprayers, by insufflators, squeeze bottles, or the like. In some aspects, nasally administered pharmaceutical compositions as disclosed herein can be deposited in the nose by medical personnel using a catheter or micropipette. In some aspects, nasally administered pharmaceutical compositions can include nasal rinses or washes for use in squeeze bottles, neti pots, or other nasal irrigation apparatuses.

In one aspect, a metered dose spray pump can dispense from about 25 to about 200 μL per spray, or about 25, 50, 75, 100, 125, 150, 175, or about 200 μL per spray, or a combination of any of the foregoing values, or a range encompassing any of the foregoing values.

In any of these aspects, various excipients can be added to nasally-delivered pharmaceutical compositions including, but not limited to, co-solvents (e.g., alcohols including ethanol, propylene glycol), osmotic pressure or tonicity regulators (e.g., dextrose, sodium chloride), buffers and pH adjustment compounds (trisodium citrate, hydrochloric acid, sodium hydroxide, sulfuric acid), preservatives (e.g., benzyl alcohol, benzalkonium chloride, chlorobutanol, methylparaben, phenylethyl alcohol, propylparaben, and related compounds), antioxidants (e.g., butylated hydroxyanisole), suspending agents and/or stabilizers (e.g., microcrystalline cellulose, sodium carboxymethyl cellulose), chelating agents (e.g. sodium EDTA), penetration enhancers (e.g., oleic acid), surfactants (e.g., PEG400, PEG 3500, polyoxyl 400 stearate, polysorbate 20, polysorbate 80), and combinations thereof.

In any of these aspects, inhaled or nasally-delivered medications can be useful if an active ingredient is poorly absorbed or degraded in the gastrointestinal tract. In a further aspect, nasal delivery may be especially useful for the delivery of peptides due to the large nasal mucosal surface area in human subjects. In still another aspect, administration via nasal delivery may enable rapid onset of action for the pharmaceutical compositions disclosed herein.

### Administration of Disclosed Compounds and Compositions

In one aspect, in the method disclosed herein, the cannabinoids or pharmaceutically acceptable salts or esters thereof is administered to the subject in the amount of from about 0.1 mg/kg to about 500 mg/kg per single dose, or at about 0.1, 0.25, 0.5, 0.75, 1, 2.5, 5, 10, 15, 25, 50, 75, 100, 125, 150, 175, 200, 225, 250, 275, 300, 325, 350, 375, 400, 425, 450, 475, or about 500 mg/kg per single dose, or a combination of any of the foregoing values, or a range encompassing any of the foregoing values.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

Unless otherwise specified, pressures referred to herein are based on atmospheric pressure (i.e. one atmosphere).

### Chemical Groups and Substituents

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as -OA¹ where A¹ is alkyl or cycloalkyl as defined above. “Alkoxy” also includes polymers of alkoxy groups as just described; that is, an alkoxy can be a polyether such as -OA¹-OA² or -OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “ Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula -NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted.

Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched)alkylene)C(O)O—N(R^{o})₂, wherein each R^{o} may be substituted as defined below and is independently hydrogen, C₁₋₆ aliphatic, —CH₂Ph, —O(CH₂)₀₋₄Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{⋅}, -(haloR^{⋅}), —(CH₂)₀₋₂OH, —(CH₂)₀₋₂OR^{⋅}, —(CH₂)₀₋₂CH(OR^{⋅})₂; —O(haloR^{⋅}), —CN, —N₃, —(CH₂)₀₋₂C(O)R^{⋅}, —(CH₂)₀₋₂C(O)OH, —(CH₂)₀₋₂C(O)OR^{⋅}, —(CH₂)₀₋₂SR^{⋅}, —(CH₂)₀₋₂SH, —(CH₂)₀₋₂NH₂, —(CH₂)₀₋₂NHR^{⋅}, —(CH₂)₀₋₂NR^{⋅}₂, —NO₂, —SiR^{⋅}₃, —OSiR^{⋅}₃, —C(O)SR^{⋅}, —(C₁₋₄ straight or branched alkylene)C(O)OR^{⋅}, or —SSR^{⋅} wherein each R^{⋅} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR^{*}₂, ═NNHC(O)R^{*}, ═NNHC(O)OR^{*}, ═NNHS(O)₂R^{*}, ═NR^{*}, ═NOR^{*}, —O(C(R^{*}₂))₂₋₃O—, or —S(C(R^{*}₂))₂₋₃S—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{*}₂)₂₋₃O—, wherein each independent occurrence of R^{*} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{*} include halogen, —R^{⋅}, -(haloR^{⋅}), —OH, —OR^{⋅}, —O(haloR^{⋅}), —CN, —C(O)OH, —C(O)OR^{⋅}, —NH₂, —NHR^{⋅}, —NR^{⋅}₂, or —NO₂, wherein each R^{⋅} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{⋅}, -(haloR^{⋅}), —OH, —OR^{⋅}, —O(haloR^{⋅}), —CN, —C(O)OH, —C(O)OR^{⋅}, —NH₂, —NHR^{⋅}, —NR^{⋅}₂, or —NO₂, wherein each R^{⋅} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and I or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

### Cannabinoids

In one aspect, the pharmaceutical compositions disclosed herein include one or more cannabinoids. Further in this aspect, the one or more cannabinoids can be natural cannabinoids produced by a plant. In an alternative aspect, the one or more cannabinoids can be synthetic or semi-synthetic cannabinoids.

In an aspect, the compositions disclosed herein can have a structure of Formula I, Formula II, Formula III, or Formula IV, a hydrogenated derivative thereof, a dehydrogenated derivative thereof, or an isomer thereof:

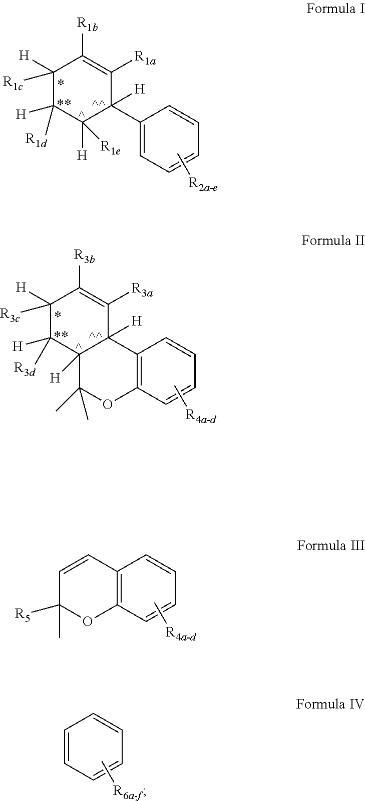

wherein when the cannabinoid has the structure of Formula I or Formula II, each R₁ or R₃ is independently selected from hydrogen, C1-C10 linear or branched alkyl, C1-C10 linear or branched alkenyl, —CH₃X, carboxyl,

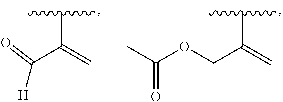

or wherein R_{1c} and R_{1c} are together a methylene bridge;

wherein X is independently selected from OH and halogen;
wherein when the cannabinoid has the structure of Formula I, Formula II, Formula III, or Formula IV, each R₂, R₄, or R₆ is independently selected from hydrogen, C1-C10 linear or branched alkyl, C1-C10 linear or branched alkenyl, carboxyl, methoxy, OH, halogen, acetoxy, —O—(CH₂)_{y}—Z, —(CH₂)_{y}—Z, or

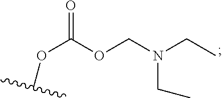

wherein y is from 1 to 10;
wherein Z is OH, CN, COOH,

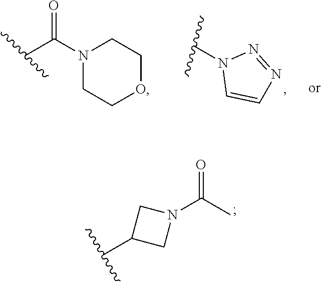

and
wherein when the cannabinoid has the structure of Formula III, R₅ is selected from C1-C10 linear or branched alkenyl.

In any of these aspects, the stereochemistry at each carbon atom indicated by *, **, \hat{ }, and/or \hat{ }\hat{ } can independently be (R) or (S).

In one aspect, the cannabinoid can include one or more of the following:

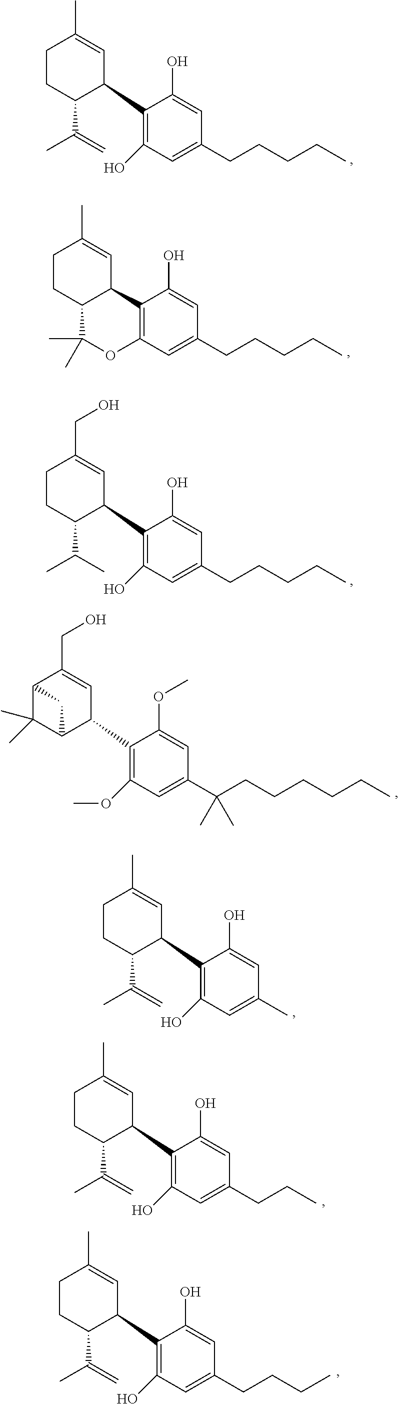

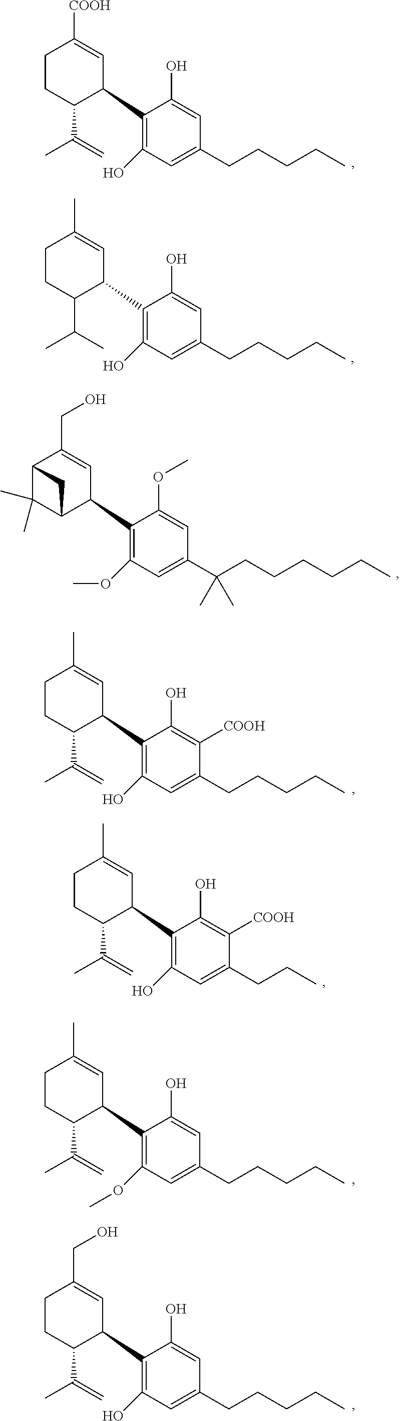

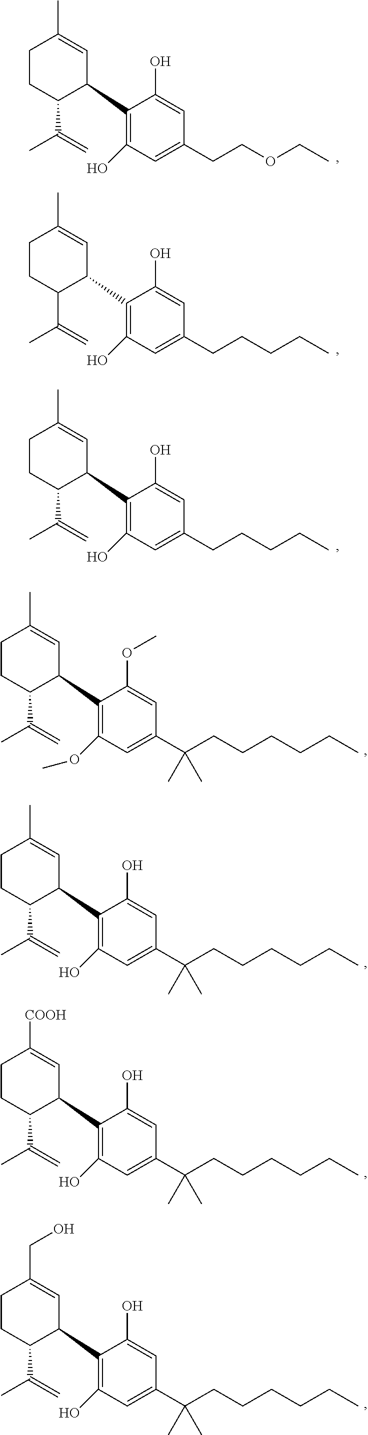

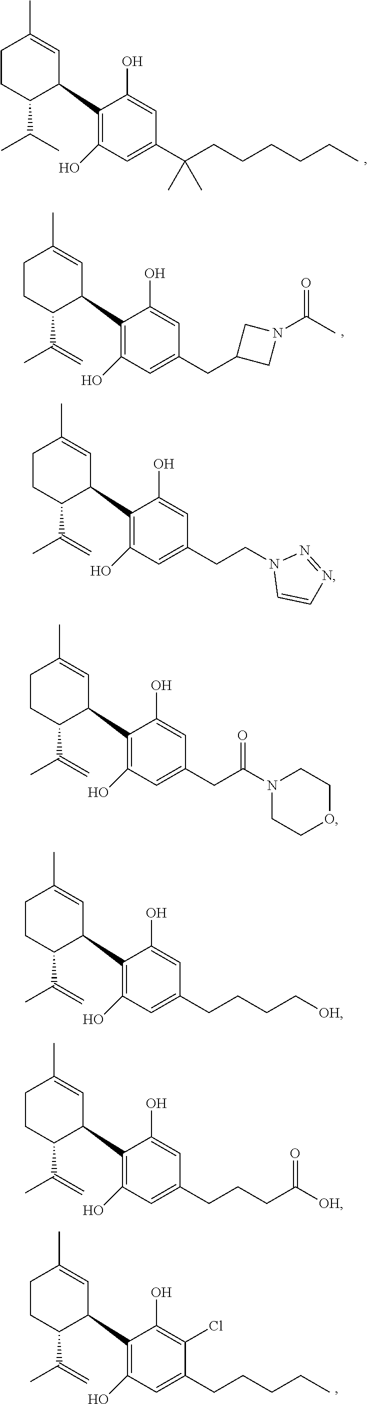

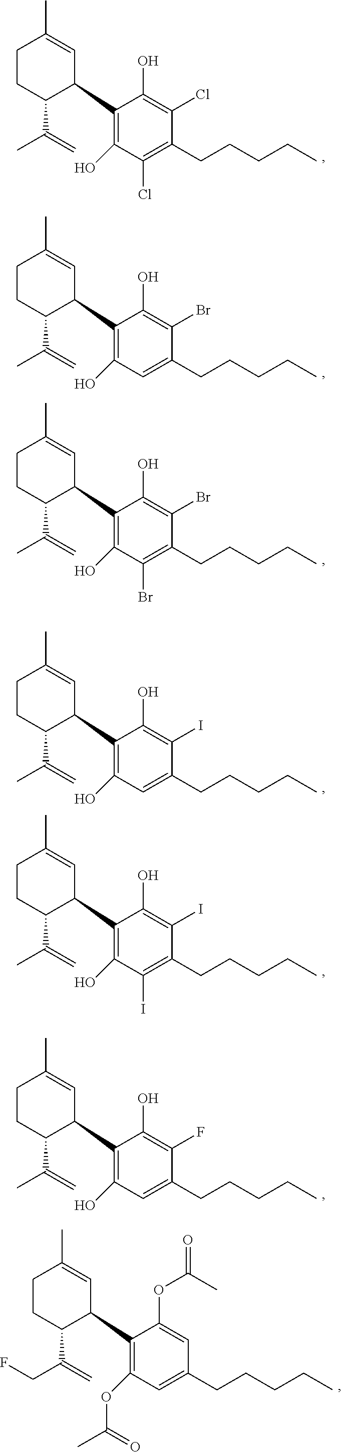

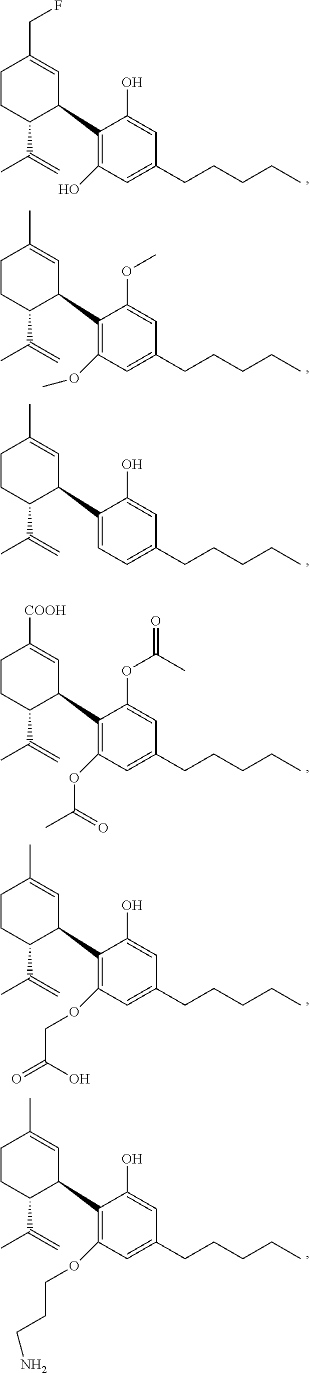

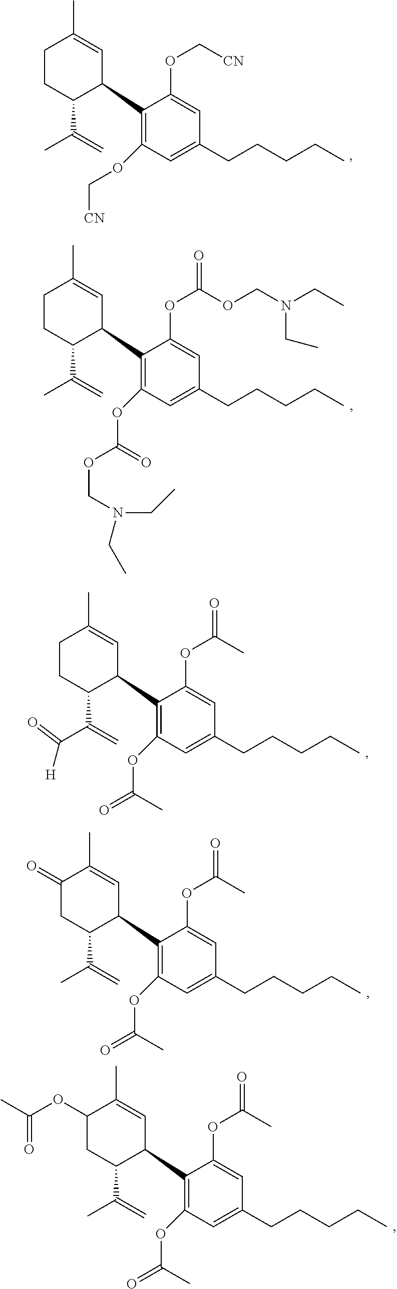

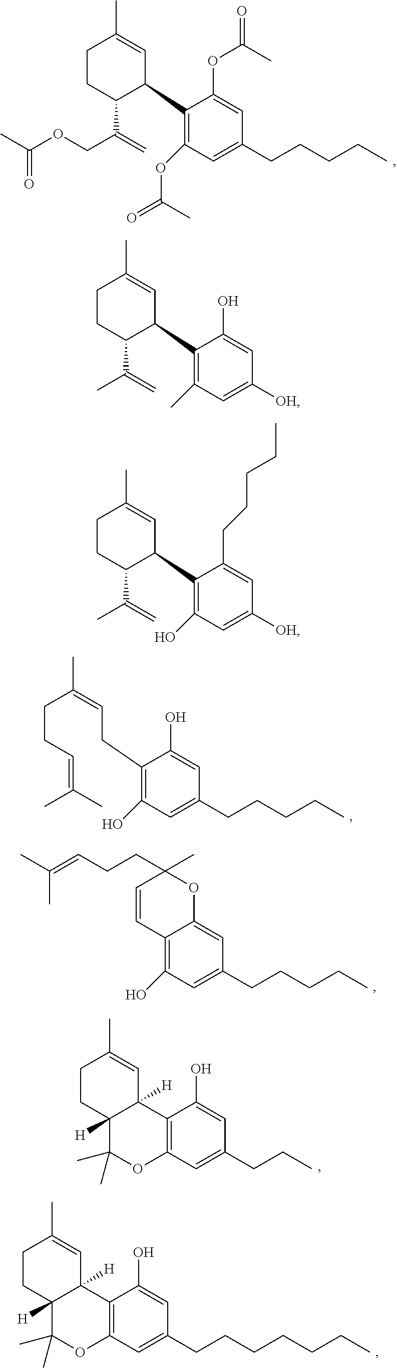

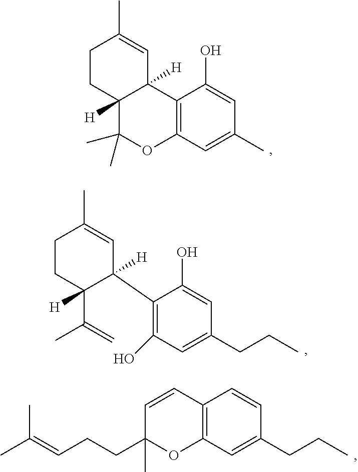

or any combination thereof.

In a further aspect, the hydrogenated derivative can be:

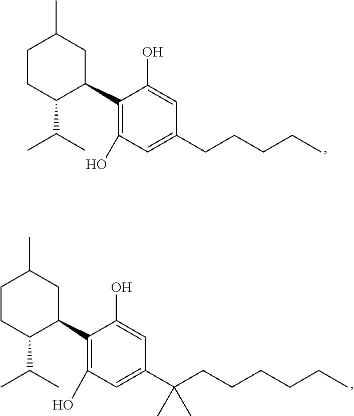 [SMILES: CCCCCc1cc(O)c([C@@H]2CC(C)CC[C@H]2C(C)C)c(O)c1 ; CCCCCCC(C)(C)c1cc(O)c([C@@H]2CC(C)CC[C@H]2C(C)C)c(O)c1]

or any combination thereof.

In one aspect, the dehydrogenated derivative can be:

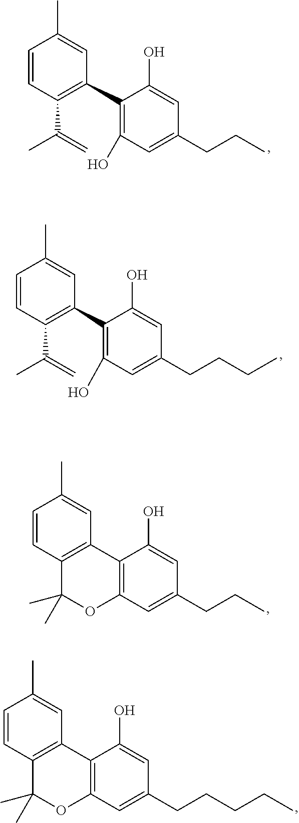

or any combination thereof.

In one aspect, the isomer is a Δ⁸ isomer and can be:

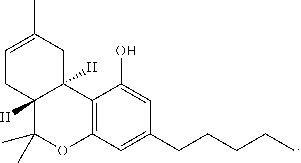 [SMILES: CCCCCc1cc(O)c2c(c1)OC(C)(C)[C@@H]1CC=C(C)C[C@@H]21]

### Aspects

Aspect 1. A pharmaceutical composition comprising (I) at least one cannabinoid or a pharmaceutically acceptable salt or ester thereof and (II) a pharmaceutically-acceptable carrier, wherein the composition is capable of being administered to a subject by inhalation.

Aspect 2. The pharmaceutical composition of Aspect 1, wherein the at least one cannabinoid comprises a structure of Formula I, Formula II, Formula III, or Formula IV, a hydrogenated derivative thereof, a dehydrogenated derivative thereof, or an isomer thereof:

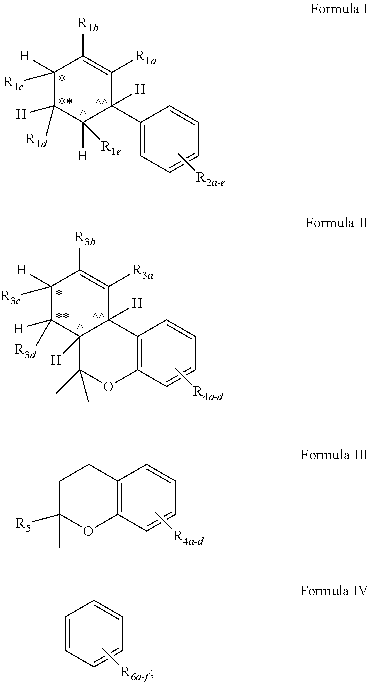

wherein when the cannabinoid has the structure of Formula I or Formula II, each R₁ or R₃ is independently selected from hydrogen, C1-C10 linear or branched alkyl, C1-C10 linear or branched alkenyl, —CH₃X, carboxyl,

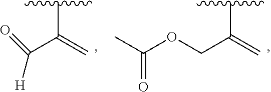

or wherein R_{1c} and R_{1c} are together a methylene bridge;
wherein X is independently selected from OH and halogen;
wherein when the cannabinoid has the structure of Formula I, Formula II, Formula III, or Formula IV, each R₂, R₄, or R₆ is independently selected from hydrogen, C1-C10 linear or branched alkyl, C1-C10 linear or branched alkenyl, carboxyl, methoxy, OH, halogen, acetoxy,

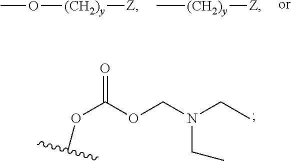

wherein y is from 1 to 10;
wherein Z is OH, CN, COOH,

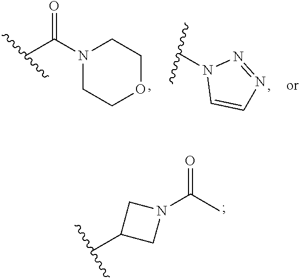

and
wherein when the cannabinoid has the structure of Formula III, R₅ is selected from C1-C10 linear or branched alkenyl.

Aspect 3. The pharmaceutical composition of Aspect 1 or 2, wherein the cannabinoid comprises

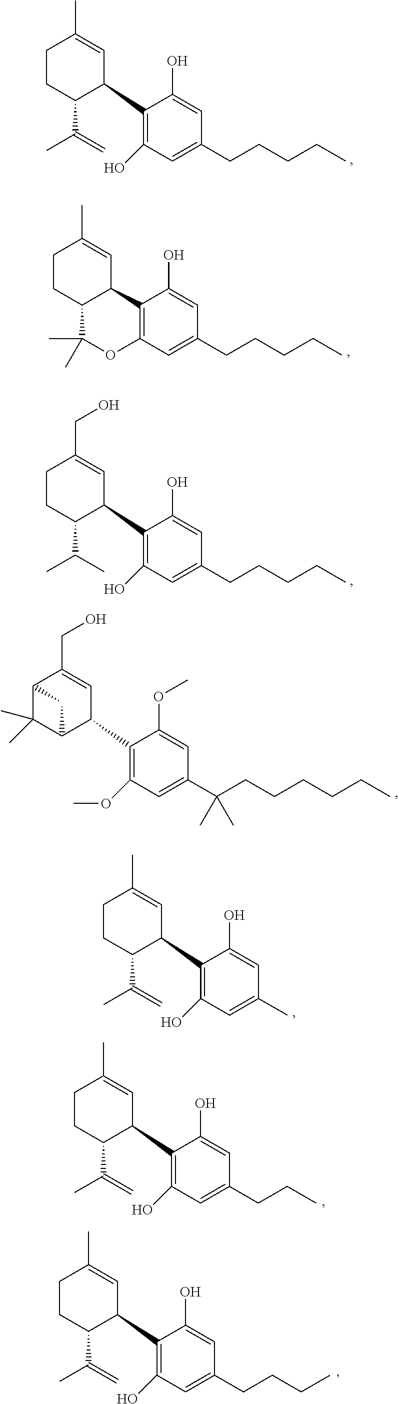

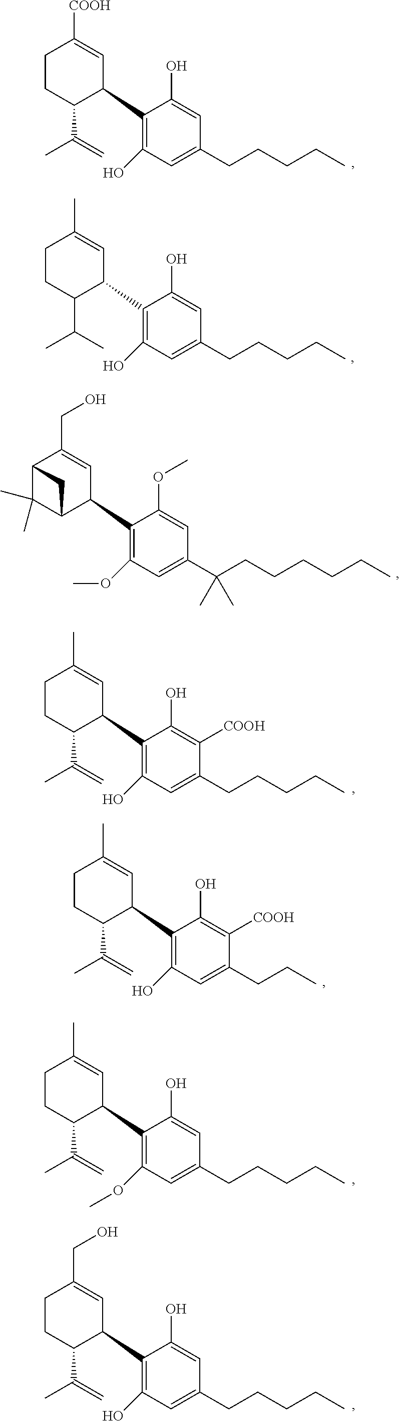

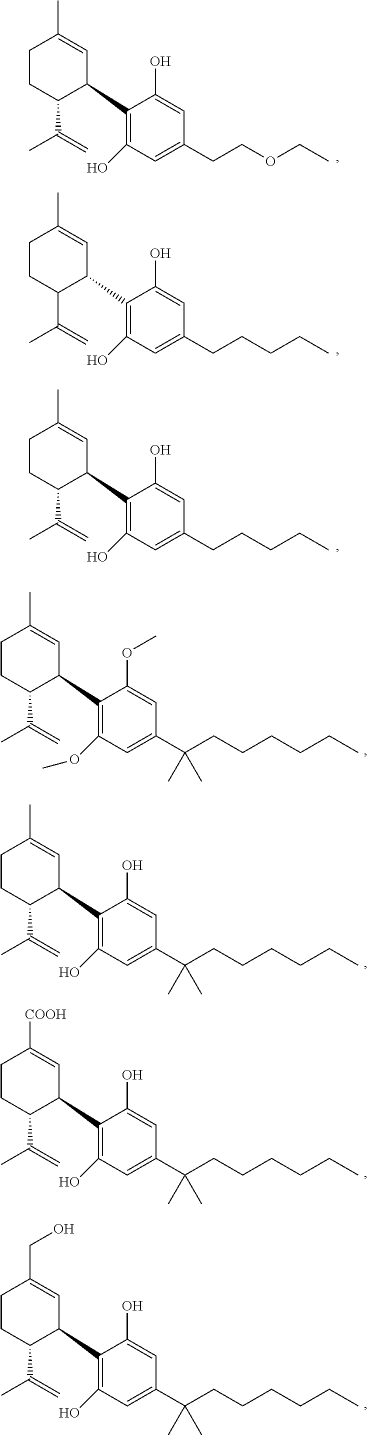

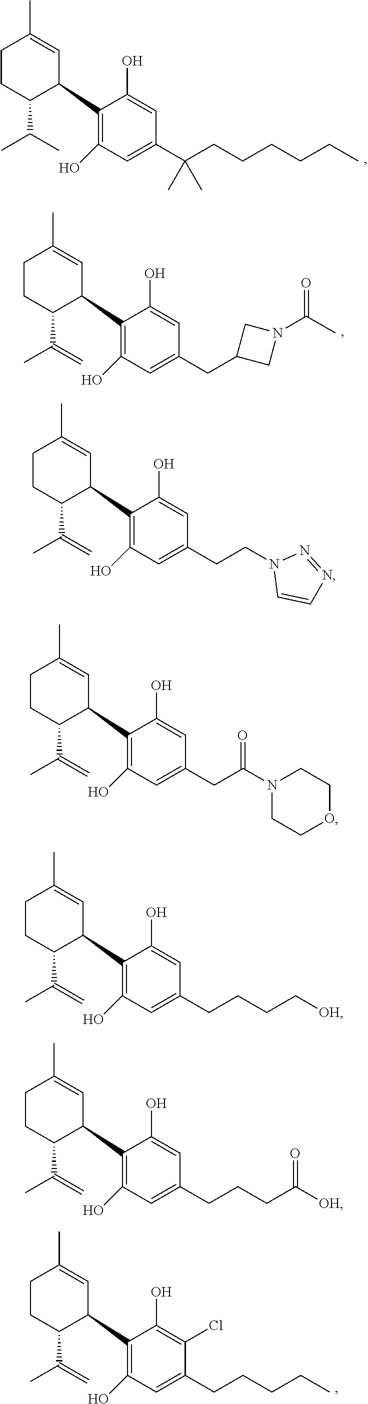

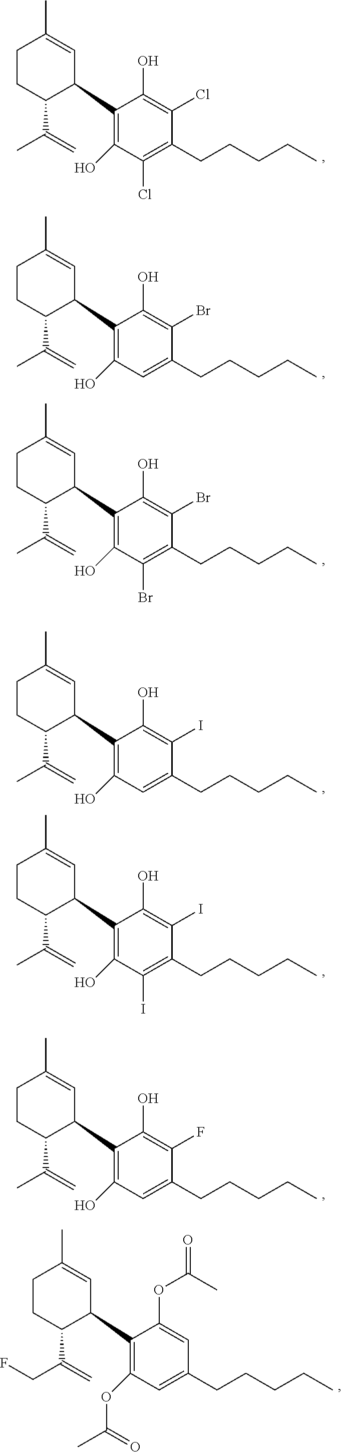

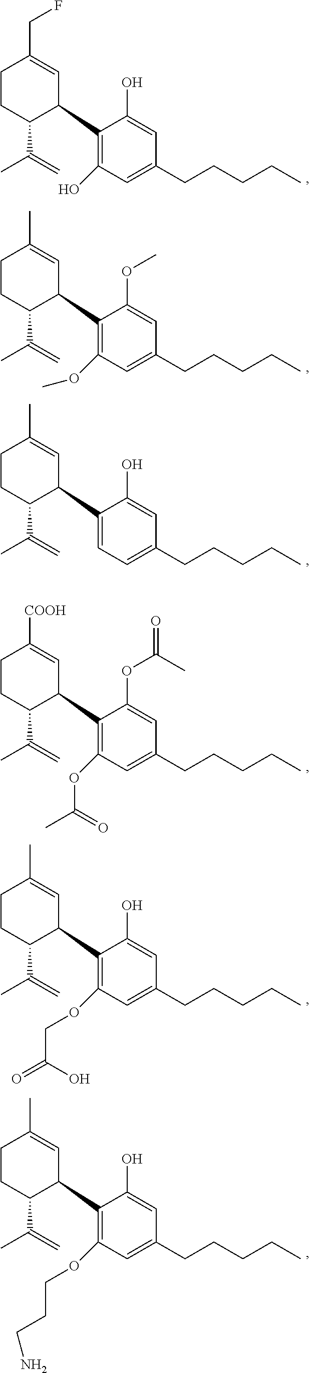

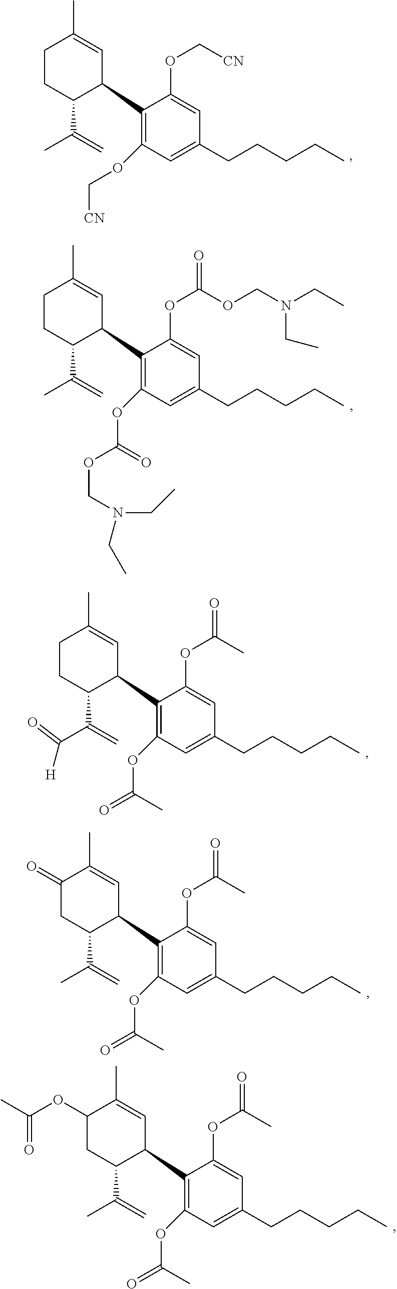

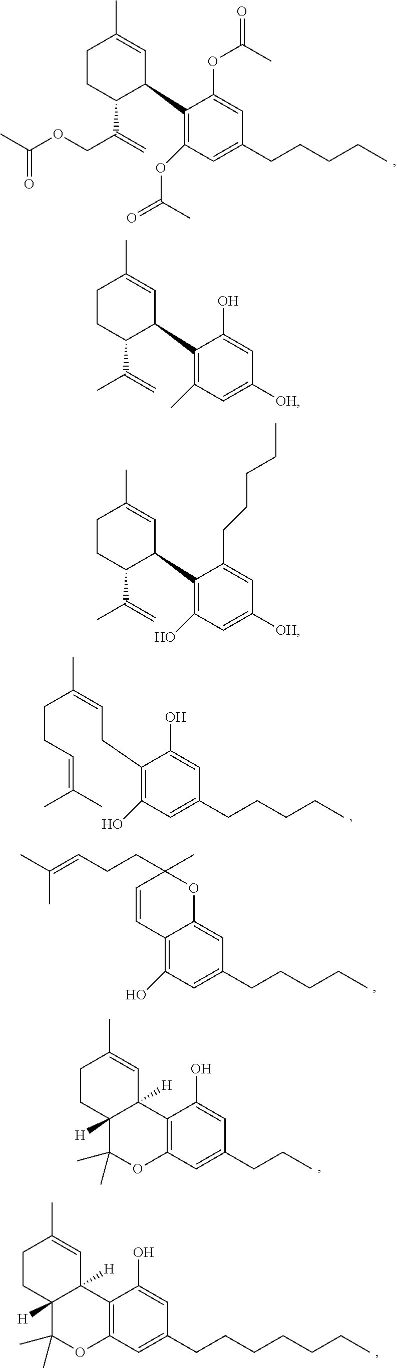

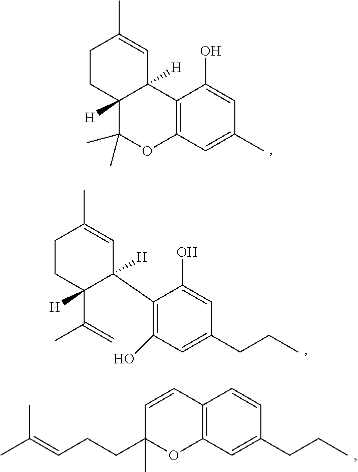

or any combination thereof.

Aspect 4. The pharmaceutical composition of Aspect 3, wherein the stereochemistry at each carbon atom indicated by *, **, \hat{ }, or \hat{ }\hat{ } is independently (R) or (S).

Aspect 5. The pharmaceutical composition of any one of Aspects 2-4, wherein the hydrogenated derivative comprises

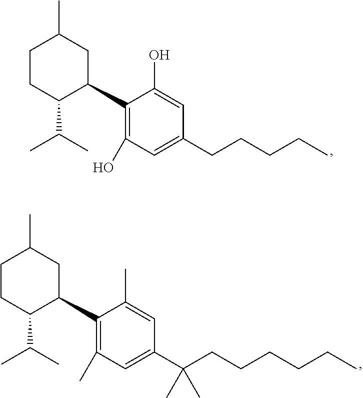

or any combination thereof.

Aspect 6. The pharmaceutical composition of any one of Aspects 2-4, wherein the dehydrogenated derivative comprises

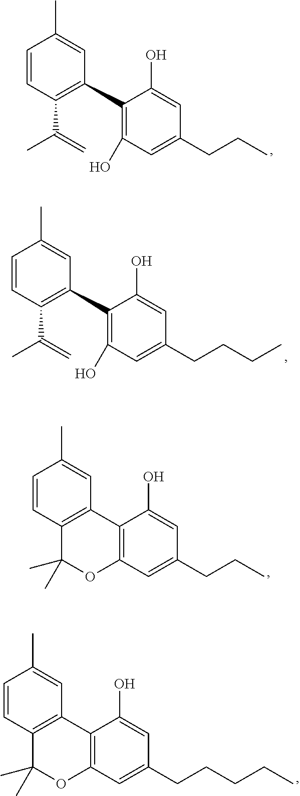

or any combination thereof.

Aspect 7. The pharmaceutical composition of any one of Aspects 2-4, wherein the isomer thereof is a Δ⁸ isomer and wherein the Δ⁸ isomer comprises

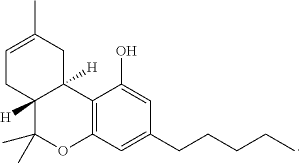 [SMILES: CCCCCc1cc(O)c2c(c1)OC(C)(C)[C@@H]1CC=C(C)C[C@@H]21]

Aspect 8. The pharmaceutical composition of any one of Aspects 1-4, wherein the cannabinoid comprises Δ⁹-tetrahydrocannabinol and cannabidiol.

Aspect 9. The pharmaceutical composition of Aspect 8, wherein the Δ⁹-tetrahydrocannabinol has a unit dose of from about 1 to about 6 mg.

Aspect 10. The pharmaceutical composition of Aspect 8 or 9, wherein the Δ⁹-tetrahydrocannabinol has a unit dose of about 3 mg.

Aspect 11. The pharmaceutical composition of any one of Aspects 8-10, wherein the cannabidiol has a unit dose of from about 1 mg to about 5 mg.

Aspect 12. The pharmaceutical composition of any one of Aspects 8-11, wherein the cannabidiol has a unit dose of from about 2.5 mg.

Aspect 13. The pharmaceutical composition of any one of Aspects 8-12, wherein the Δ⁹-tetrahydrocannabinol and the cannabidiol are present in a weight ratio of from about 1:5 to about 5:1.

Aspect 14. The pharmaceutical composition of any one of Aspects 8-13, wherein the Δ⁹-tetrahydrocannabinol and the cannabidiol are present in a weight ratio of about 3:2.5.

Aspect 15. The pharmaceutical composition of any one of Aspects 1-4, wherein the cannabinoid comprises Δ⁹-tetrahydrocannabinol and cannabichromene.

Aspect 16. The pharmaceutical composition of Aspect 15, wherein the Δ⁹-tetrahydrocannabinol has a unit dose of from about 1 to about 6 mg.

Aspect 17. The pharmaceutical composition of Aspect 15 or 16, wherein the Δ⁹-tetrahydrocannabinol has a unit dose of about 3 mg.

Aspect 18. The pharmaceutical composition of any one of Aspects 15-17, wherein the cannabichromene has a unit dose of from about 1 mg to about 5 mg.

Aspect 19. The pharmaceutical composition of any one of Aspects 15-18, wherein the cannabichromene has a unit dose of from about 2.3 mg.

Aspect 20. The pharmaceutical composition of any one of Aspects 15-19, wherein the Δ⁹-tetrahydrocannabinol and the cannabichromene are present in a weight ratio of from about 1:5 to about 5:1.

Aspect 21. The pharmaceutical composition of any one of Aspects 15-20, wherein the Δ⁹-tetrahydrocannabinol and the cannabichromene are present in a weight ratio of about 3:2.3.

Aspect 22. The pharmaceutical composition of any one of the preceding Aspects, wherein the pharmaceutically acceptable salt comprises an organic salt, a metal salt, or any combination thereof.

Aspect 23. The pharmaceutical composition of any one of the preceding Aspects, wherein the pharmaceutically acceptable salt comprises NH⁴⁺, Na⁺, Li⁺, K⁺, Ca²⁺, Mg²⁺, Fe²⁺, Fe²⁺, Cu²⁺, Al³⁺, Zn²⁺, 2-trimethylethanolammonium cation (choline), or a quaternary salt of isopropylamine, trimethylamine, diethylamine, triethylamine, tripropylamine, ethanolamine, 2-dimethylaminoethanol, 2-diethylaminoethanol, lysine, arginine, histidine, or any combination thereof.

Aspect 24. The pharmaceutical composition of any one of the preceding Aspects, wherein the pharmaceutically acceptable ester is a prodrug.

Aspect 25. The pharmaceutical composition of any one of the preceding Aspects, further comprising peppermint oil, peppermint extract, or any combination thereof.

Aspect 26. The pharmaceutical composition of any one of the preceding Aspects, wherein the composition does not include caffeine.

Aspect 27. The pharmaceutical composition of any one of the preceding Aspects, wherein the composition comprises particles having an average diameter of about 9 μm or less.

Aspect 28. The pharmaceutical composition of any one of Aspects 1-27, wherein the pharmaceutical composition comprises a spray comprising from about 20 mg to about 60 mg of ethanol per 100 μL of spray.

Aspect 29. The pharmaceutical composition of any one of Aspects 1-27, wherein the pharmaceutical composition comprises a spray comprising:
a. Δ⁹-tetrahydrocannabinol in an amount of from about 1 mg to about 5 mg per 100 μL of spray; and
b. cannabidiol or cannabichromene in an amount of from about 1 mg to about 5 mg per 100 μL of spray.

Aspect 30. The pharmaceutical composition of any one of Aspects 1-27, wherein the pharmaceutical composition comprises a spray comprising:
a. Δ⁹-tetrahydrocannabinol in an amount of from about 1 mg to about 5 mg per 100 μL of spray;
b. cannabidiol or cannabichromene in an amount of from about 1 mg to about 5 mg per 100 μL of spray; and
c. ethanol in an amount of from about 20 mg to about 60 mg per 100 μL of spray.

Aspect 31. The pharmaceutical composition of any one of the preceding Aspects, wherein the pharmaceutically-acceptable carrier comprises an inhalable dry powder.

Aspect 32. A dry powder inhaler comprising the pharmaceutical composition of Aspect 31.

Aspect 33. An inhaler comprising the pharmaceutical composition of any one of Aspects 1-27.

Aspect 34. A metered-dose nasal pump spray comprising the pharmaceutical composition in any one of Aspects 1-27.

Aspect 35. The pharmaceutical composition of any one of Aspects 1-27, wherein the pharmaceutically-acceptable carrier comprises saline or sterile water.

Aspect 36. A nebulizer ampule comprising the pharmaceutical composition of Aspect 35.

Aspect 37. The method of any one of Aspects 1-27, wherein the pharmaceutical composition is formulated as a 100 μL spray.

Aspect 38. The method of Aspect 37, wherein the pharmaceutically-acceptable carrier comprises ethanol.

Aspect 39. The method of Aspect 37, wherein the ethanol is present in a volume of about 51 μL.

Aspect 40. A method for treating or preventing at least one symptom associated with migraine in a subject, the method comprising administering to the subject the pharmaceutical composition of any one of Aspects 1-27.

Aspect 41. The method of Aspect 40, wherein the at least one symptom comprises sensitivity to light, sensitivity to sound, nausea, vomiting, pain, weakness, numbness, vision loss, difficulty speaking, visual hallucinations, or any combination thereof.

Aspect 42. The method of Aspect 40 or 41, wherein the subject is a human.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Formulations

Exemplary formulations according to the present disclosure are shown in Table 1:

TABLE 1
Spray Formulations
Formulation 1

Component

| | Δ⁹-Tetrahydrocannabinol | Cannabidiol |
|---|---|---|
| Amount | 3 mg | 2.5 mg |

Formulation 2

Component

| | Δ⁹-Tetrahydrocannabinol | Cannabichromene |
|---|---|---|
| Amount | 3 mg | 2.3 mg |

Cannabinoids in these spray formulations were isolated and purified from Cannabis sativa L. For each 100 μL of the formulation, up to 40 mg of ethanol can be included, for a total of about 51 μL ethanol per 100 μL.

Some formulations additionally included a peppermint oil or extract from Mentha×piperita.

What is claimed is:

1. A pharmaceutical composition comprising (I) at least one cannabinoid or a pharmaceutically acceptable salt or ester thereof and (II) a pharmaceutically-acceptable carrier, wherein the composition is capable of being administered to a subject by inhalation.

2. The pharmaceutical composition of claim 1, wherein the at least one cannabinoid comprises a structure of Formula I, Formula II, Formula III, or Formula IV, a hydrogenated derivative thereof, a dehydrogenated derivative thereof, or an isomer thereof: 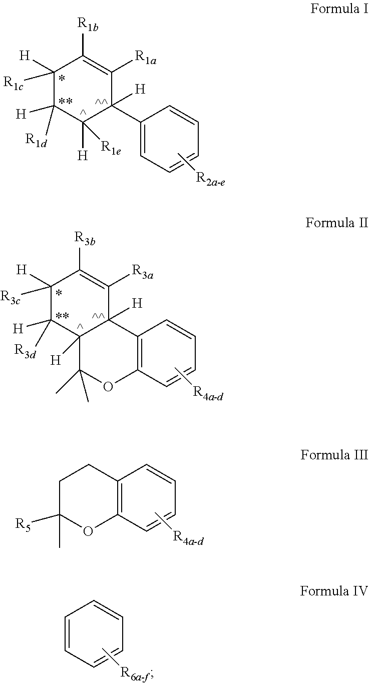
wherein when the cannabinoid has the structure of Formula I or Formula II, each R₁ or R₃ is independently selected from hydrogen, C1-C10 linear or branched alkyl, C1-C10 linear or branched alkenyl, —CH₃X, carboxyl, 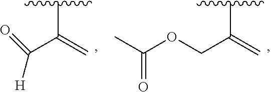
or wherein R_{1c} and R_{1c} are together a methylene bridge;
wherein X is independently selected from OH and halogen;
wherein when the cannabinoid has the structure of Formula I, Formula II, Formula III, or Formula IV, each R₂, R₄, or R₆ is independently selected from hydrogen, C1-C10 linear or branched alkyl, C1-C10 linear or branched alkenyl, carboxyl, methoxy, OH, halogen, acetoxy, —O—(CH₂)_{y}—Z, —(CH₂)_{y}—Z, or 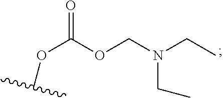
wherein y is from 1 to 10;
wherein Z is OH, CN, COOH, 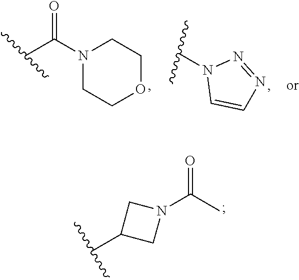
and wherein when the cannabinoid has the structure of Formula III, R₅ is selected from C1-C10 linear or branched alkenyl.

3. The pharmaceutical composition of claim 1, wherein the cannabinoid comprises 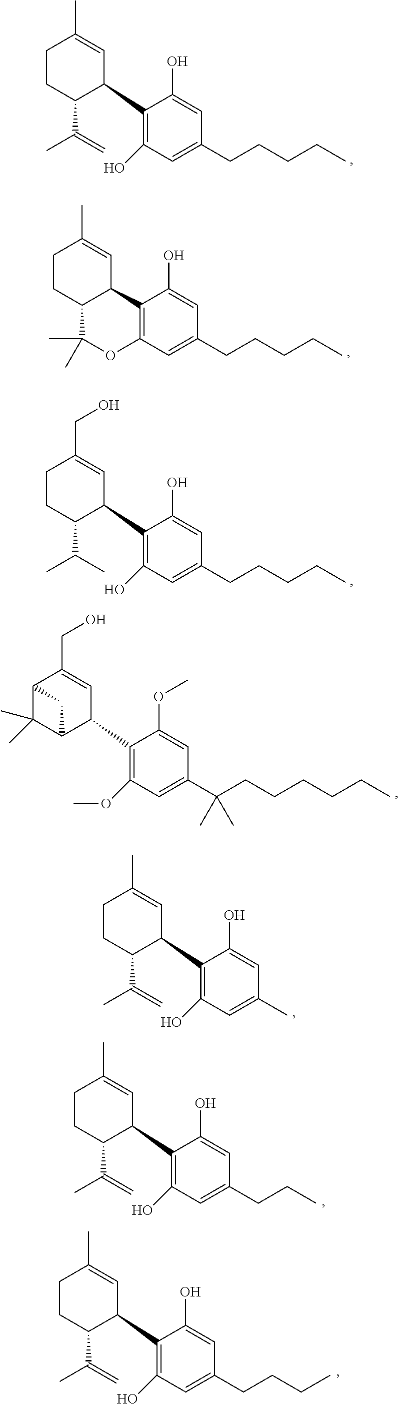 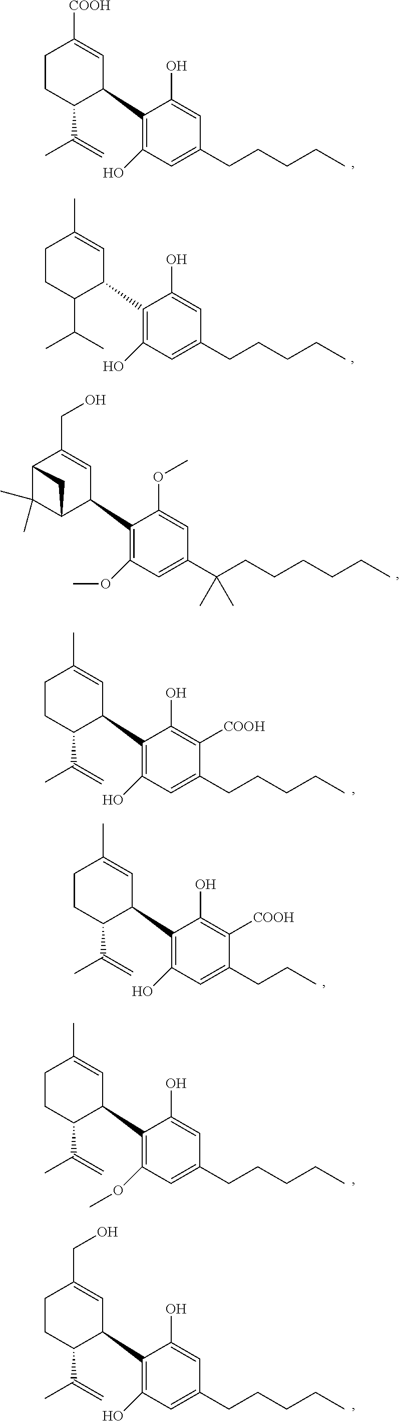 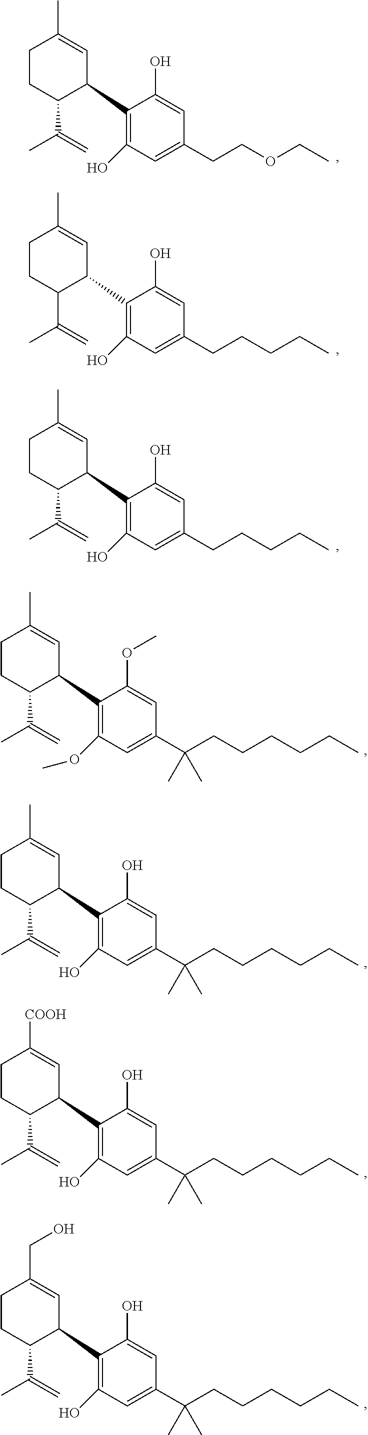 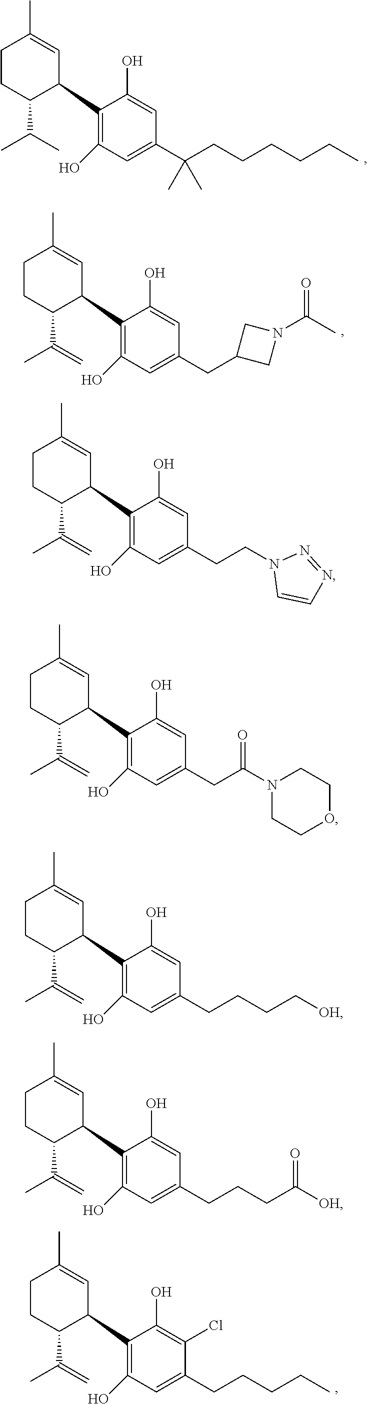 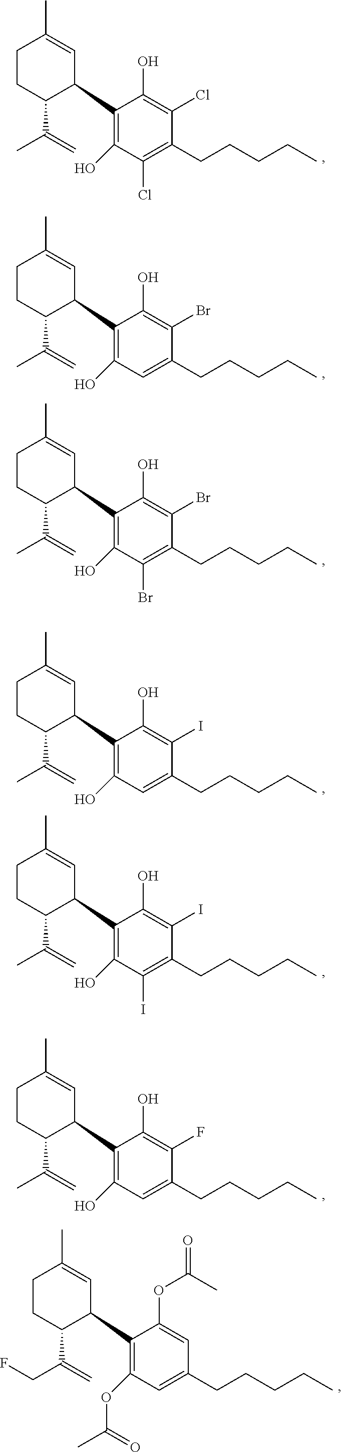 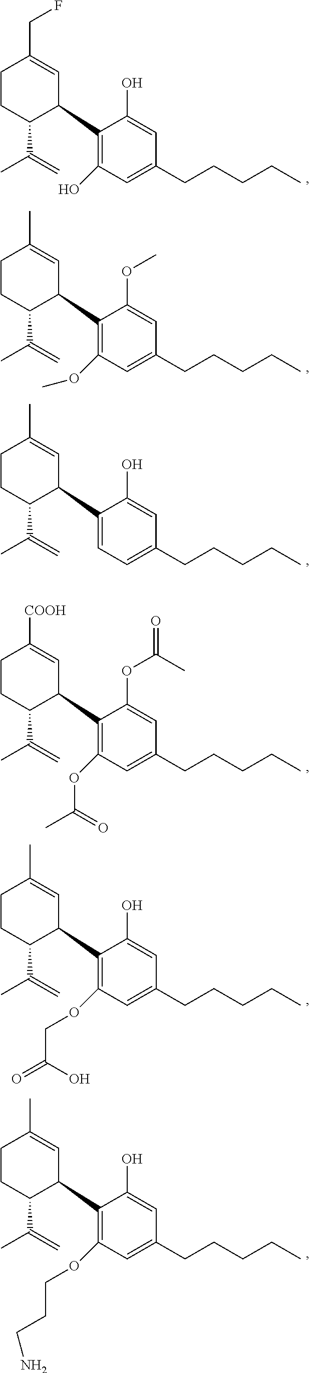 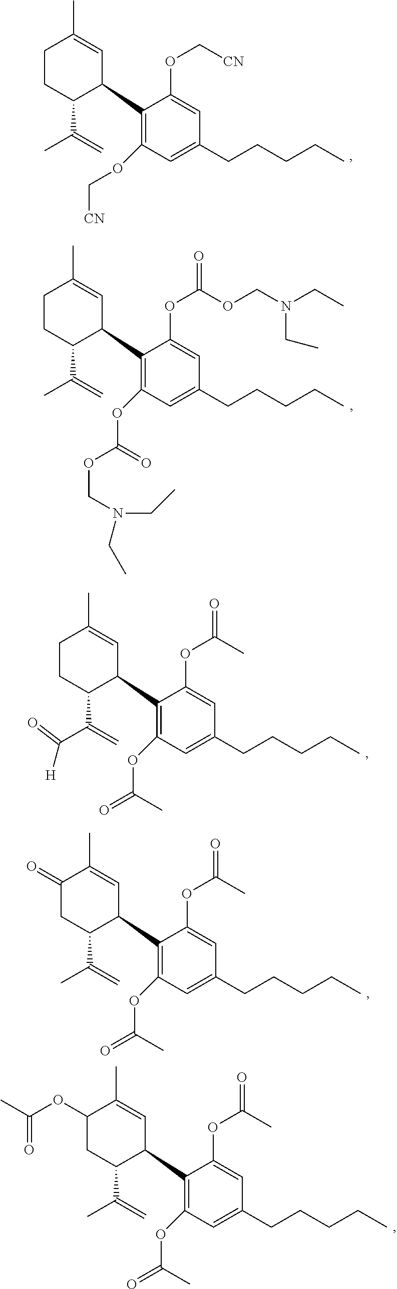 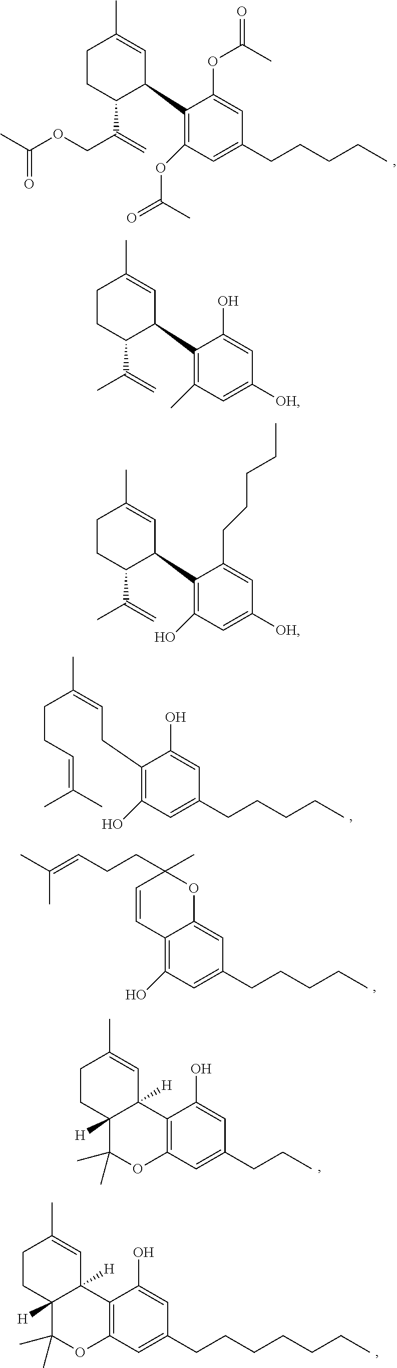 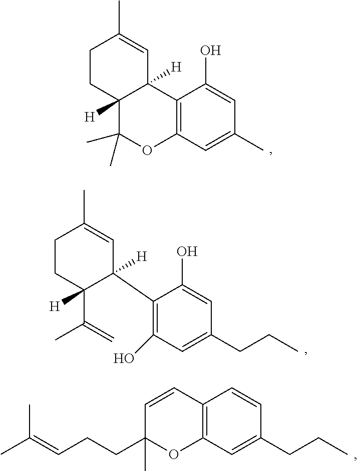
or any combination thereof.

4. The pharmaceutical composition of claim 3, wherein the stereochemistry at each carbon atom indicated by *, **, \hat{ }, or \hat{ }\hat{ } is independently (R) or (S).

5. The pharmaceutical composition of claim 2, wherein the hydrogenated derivative comprises 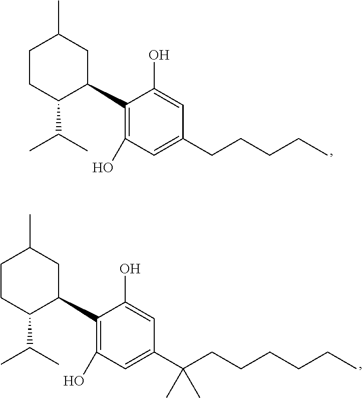 [SMILES: CCCCCc1cc(O)c([C@@H]2CC(C)CC[C@H]2C(C)C)c(O)c1 ; CCCCCCC(C)(C)c1cc(O)c([C@@H]2CC(C)CC[C@H]2C(C)C)c(O)c1]
or any combination thereof.

6. The pharmaceutical composition of claim 2, wherein the dehydrogenated derivative comprises 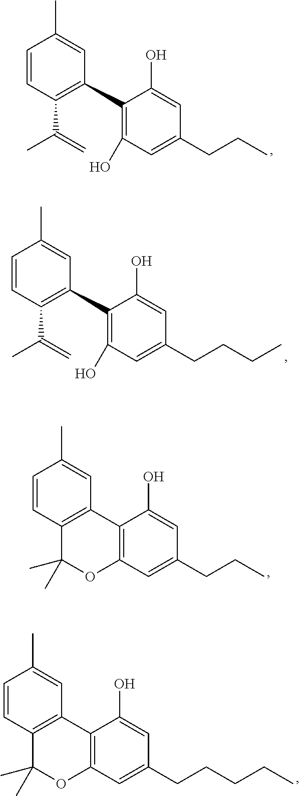
or any combination thereof.

7. The pharmaceutical composition of claim 2, wherein the isomer thereof is a Δ⁸ isomer and wherein the Δ⁸ isomer comprises

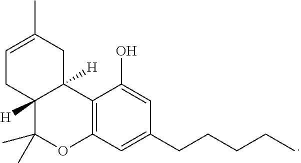 [SMILES: CCCCCc1cc(O)c2c(c1)OC(C)(C)[C@@H]1CC=C(C)C[C@@H]21]

8. The pharmaceutical composition of claim 1, wherein the cannabinoid comprises Δ⁹-tetrahydrocannabinol and cannabidiol.

9. The pharmaceutical composition of claim 8, wherein the Δ⁹-tetrahydrocannabinol has a unit dose of from about 1 to about 6 mg.

10. The pharmaceutical composition of claim 8, wherein the cannabidiol has a unit dose of from about 1 mg to about 5 mg.

11. The pharmaceutical composition of claim 8, wherein the Δ⁹-tetrahydrocannabinol and the cannabidiol are present in a weight ratio of from about 1:5 to about 5:1.

12. The pharmaceutical composition of claim 1, wherein the cannabinoid comprises Δ⁹-tetrahydrocannabinol and cannabichromene.

13. The pharmaceutical composition of claim 12, wherein the Δ⁹-tetrahydrocannabinol has a unit dose of from about 1 to about 6 mg.

14. The pharmaceutical composition of claim 12, wherein the cannabichromene has a unit dose of from about 1 mg to about 5 mg.

15. The pharmaceutical composition of claim 12, wherein the Δ⁹-tetrahydrocannabinol and the cannabichromene are present in a weight ratio of from about 1:5 to about 5:1.

16. The pharmaceutical composition of claim 1, wherein the pharmaceutical composition comprises a spray comprising from about 20 mg to about 60 mg of ethanol per 100 μL of spray.

17. The pharmaceutical composition of claim 1, wherein the pharmaceutical composition comprises a spray comprising:
(a) Δ⁹-tetrahydrocannabinol in an amount of from about 1 mg to about 5 mg per 100 μL of spray;
(b) cannabidiol or cannabichromene in an amount of from about 1 mg to about 5 mg per 100 μL of spray; and
(c) ethanol in an amount of from about 20 mg to about 60 mg per 100 μL of spray.

18. The pharmaceutical composition of claim 1, wherein the pharmaceutically-acceptable carrier comprises an inhalable dry powder.

19. An inhaler, metered-dose nasal pump spray, or a nebulizer ampule comprising the pharmaceutical composition of claim 1.

20. A method for treating or preventing at least one symptom associated with migraine in a subject, the method comprising administering to the subject the pharmaceutical composition of claim 1.
